(19) United States
(12) Patent Application Publication
(10) Pub. No.: US 2015/0119785 A1
(43) Pub. Date: Apr. 30, 2015

(54) EAR WAX CLEANING DEVICE

(71) Applicant: TERATA ANN KANU, OKLAHOMA CITY, OK (US)

(72) Inventor: TERATA ANN KANU, OKLAHOMA CITY, OK (US)

(21) Appl. No.: 14/172,716

(22) Filed: Feb. 4, 2014

Related U.S. Application Data
(63) Continuation-in-part of application No. 14/067,860, filed on Oct. 30, 2013.

(51) Int. Cl. A61F 13/40

(52) U.S. Cl. CPC A61M 35/006
USPC 604/3

(57) ABSTRACT

Some embodiments of the present invention include a device for cleaning earwax from an ear canal, the device having a tube with a distal end and a proximal end, an absorbent tip attached to the distal end of the tube, a bulb attached to the proximal end of the tube, and a volume of hydrogen peroxide positioned within the bulb. The tube may be sealed with a diaphragm preventing the hydrogen peroxide from flowing down the tube into the absorbent tip. The diaphragm may be capable of rupturing upon an external force being applied to the bulb causing the cleaning solution to flow from the bulb through the tube and into the absorbent tip. A length of the tube may be shorter than an average adult's external ear canal.

### RELATED APPLICATION

This application claims priority to non-provisional patent application U.S. Ser. No. 14/067,860 filed on Oct. 30, 2013, the entire contents of which is herein incorporated by reference.

### BACKGROUND

The embodiments herein relate generally to toiletries, and more particularly, to a device for cleaning cerumen, more commonly known as earwax, from the ear canal.

Earwax, germs, and bacteria tend to build up in the ear canal. Cotton swabs are probably the most well-known product used for cleaning the ear and removing earwax and other unwanted materials from the external ear canals. However, users have a tendency to insert the cotton swab too far into their ear canal, damaging the tympanic membrane, more commonly known as the ear drum, and sometimes even cause a perforation in the ear drum, which can have long term sequelae. Additionally, traditional cotton swabs can cause earwax impaction in the ear canal instead of removing the earwax. As a result, many cotton swab manufacturers print a warning on the packaging instructing users not to insert the swab in the ear canal.

Therefore, what is needed is a device for cleaning the outer and external ear canals with a lowered risk of perforating the ear drum.

### SUMMARY

Some embodiments of the present invention include a device for cleaning earwax from an ear canal, the device having a tube with a distal end and a proximal end, an absorbent tip attached to the distal end of the tube, a bulb attached to the proximal end of the tube, and a volume of hydrogen peroxide positioned within the bulb. The tube may be sealed with a diaphragm preventing the hydrogen peroxide from flowing down the tube into the absorbent tip. The diaphragm may be capable of rupturing upon an external force being applied to the bulb causing the cleaning solution to flow from the bulb through the tube and into the absorbent tip. A length of the tube may be shorter than an average adult's external ear canal.

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of one embodiment of the present invention.

FIG. 2 is a side elevation view of one embodiment of the present invention with parts broken away.

FIG. 3 is a side elevation view of one embodiment of the present invention with parts broken away.

FIG. 4 is a cross-sectional view of an ear canal with one embodiment of the present invention in use.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to clean earwax from the ear canal and may comprise the following elements. This list of possible constituent elements is intended to be exemplary only, and it is not intended that this list be used to limit the system of the present application to just these elements. Persons having ordinary skill in the art relevant to the present disclosure may understand there to be equivalent elements that may be substituted within the present disclosure without changing the essential function or operation of the system.

1. Bulb

2. Tube

3. Absorbent Tip

The various elements of the device for cleaning an ear canal of the present disclosure may be related in the following exemplary fashion. It is not intended to limit the scope or nature of the relationships between the various elements and the following examples are presented as illustrative examples only.

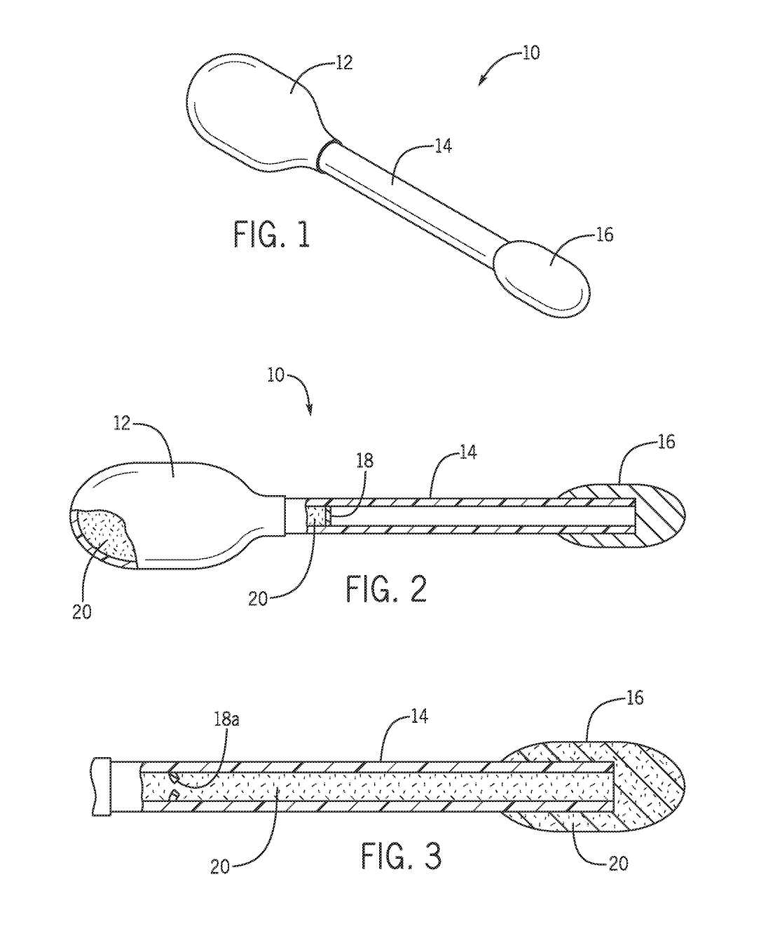

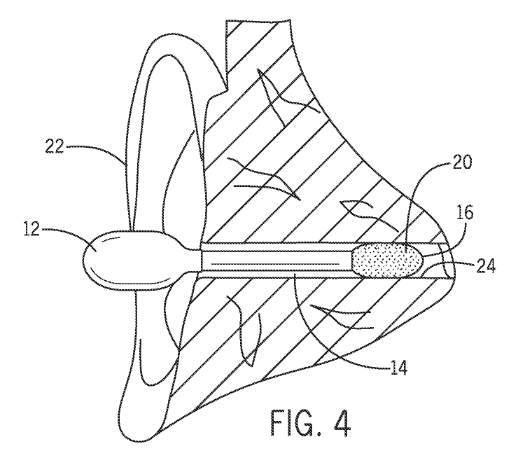

By way of example, and referring to FIG. 1, one embodiment of the present device 10 comprises a tube 14 having a proximal end and a distal end, the distal end being attached to an absorbent tip 16 and the proximal end being connected to a bulb 12. As shown in FIG. 2, the bulb 16 may be configured to hold a volume of a cleaning solution 20, and the cleaning solution 20 may be prevented from flowing into the tube 14 by a diaphragm 18. When the bulb 16 is squeezed or depressed, the diaphragm 18 may rupture. As shown in FIG. 3, the ruptured diaphragm 18a allows for the cleaning solution 20 to travel from the bulb 12, through the tube 14, and into the absorbent tip 16, into which the tube 14 extends. The cleaning solution 20 may distribute throughout the absorbent tip 16. As shown in FIG. 4, a user may insert the proximal end of the tube 14 having the absorbent tip 16 into the ear canal 24, and the cleaning solution 20 may be applied to the ear canal 24 from the absorbent tip 16.

As shown in FIGS. 1 and 2, the proximal end of tube 14 may insert into bulb 12. In embodiments, the bulb 12 is hollow and has an opening into which the tube 14 fits snugly. The tube 14 may fit tightly enough into the opening of the bulb 12 to prevent the cleaning solution from leaking out of the device proximal to the opening of the bulb 12. The bulb may be made of suitable materials and, in some embodiments, is made of rubber. The bulb may be designed to hole a volume of cleaning solution. For example, in embodiments, the bulb may hold from about 1 to about 3 mL of the cleaning solution. However, the bulb may be scaled up or down to hold a larger or smaller volume of cleaning solution.

In embodiments, the tube is a hollow shaft. The tube may be made of any suitable material and, in embodiments, is made of a plastic or paper material. The tube may have a length equal to or less than the length of an average adult's external ear canal in order to lower the likelihood of perforating the ear drum. The average adult's external ear canal is typically about one inch (2.54 cm) long. For example, the tube may have a length of about one inch or smaller. In embodiments, the tube is one inch (or 2.54 cm) long. However, the length of the tube may be varied. For example, the tube may have a shorter length if being used in a child.

As shown in FIGS. 2 and 3, the proximal end of the tube 14 may have a diaphragm 18. When the bulb is squeezed or depressed, the diaphragm 18 ruptures, as shown in FIG. 3. The diaphragm may be made of any suitable material, such as plastic or a paper material. Suitable diaphragm materials include those materials capable of rupturing when the bulb is squeezed, but also capable of remaining intact during normal handling of the device.

When the diaphragm 18 ruptures, the cleaning solution 20 travels through the tube into the absorbent tip 16. The absorbent tip may be made of any suitable material and, in embodiments, is a cotton swab made of tightly wound cotton, such as that used to make conventional cotton swab tips.

The cleaning solution may be any liquid solution capable of disinfecting and cleaning dirt or grime. For example, when the device is used to clean a human ear canal, the cleaning solution may be hydrogen peroxide. In other embodiments, the cleaning solution may be rubbing alcohol or any other conventional disinfectant. For example, if the device is used for removing make-up, the cleaning solution may be make-up remover or nail polish remover.

In embodiments, a user may use the device to clean the external ear canal, disinfect wounds, remove make up, or for any other task that cotton swabs are conventionally used.

In embodiments, when a user places the absorbent tip containing cleaning solution into his or her ear, the absorbent tip may be used to clean the ear canal, removing earwax and other germs and bacteria. Because the length of the tube is the same as or less than the average length of an external ear canal, the risk of perforating the ear drum is lower than in conventional cotton swabs. In other embodiments, a user may squeeze the bulb and use the absorbent tip that is saturated with the cleaning solution to disinfect a cut or scrape.

Persons of ordinary skill in the art may appreciate that numerous design configurations may be possible to enjoy the functional benefits of the inventive device. Thus, given the wide variety of configurations and arrangements of embodiments of the present invention the scope of the invention is reflected by the breadth of the claims below rather than narrowed by the embodiments described above.

What is claimed is:

1. A cleaning device for holding and applying a cleaning solution, the device comprising:
a tube having a distal end and a proximal end;
an absorbent tip attached to the distal end of the tube;
a bulb attached to the proximal end of the tube; and
a volume of cleaning solution temporarily stored within the bulb, the tube being sealed with a diaphragm preventing the cleaning solution from flowing down the tube into the absorbent tip,
wherein:
the diaphragm is capable of rupturing upon an external force being applied to the bulb, causing the cleaning solution to flow from the bulb through the tube and into the absorbent tip, saturating the absorbent tip.

2. The device of claim 1, wherein the tube has a length of about 1 inch.

3. The device of claim 1, wherein the volume of cleaning solution is from about 1 mL to about 3 mL.

4. The device of claim 1, wherein the cleaning solution is capable of breaking down earwax.

5. The device of claim 1, wherein the absorbent tip is a cotton swab tip.

6. A device for cleaning earwax from an ear canal, the device comprising:
a tube having a distal end and a proximal end;
an absorbent tip attached to the distal end of the tube;
a bulb attached to the proximal end of the tube; and
a volume of hydrogen peroxide temporarily stored within the bulb, the tube being sealed with a diaphragm preventing the hydrogen peroxide from flowing down the tube into the absorbent tip,
wherein:
the diaphragm is capable of rupturing upon an external force being applied to the bulb causing the cleaning solution to flow from the bulb through the tube and into the absorbent tip; and
a length of the tube is shorter than an average adult's external ear canal.

7. The device of claim 6, wherein the length of the tube is about 1 inch or less.

8. The device of claim 6, wherein the volume of hydrogen peroxide is from about 1 mL to about 3 mL.

9. The device of claim 6, wherein the absorbent tip is a cotton swab tip.

10. The device of claim 6, wherein:
the bulb has an opening into which the proximal end of the tube fits;
the tube fits tightly enough into the opening in the bulb to prevent hydrogen peroxide from leaking out of the device proximal to the opening of the bulb; and
the absorbent tip is wound around the distal end of the tube.
